(19) United States
(12) Patent Application Publication
(10) Pub. No.: US 2011/0054299 A1
(43) Pub. Date: Mar. 3, 2011

(54) Method, System, and Computer-Accessible Medium for Assessment of Glycosaminoglycan Concentration in Vivo by Chemical Exchange Saturation Transfer

(76) Inventors: Wen Ling, Minneapolis, MN (US); Ravinder R. Regatte, Monroe, NJ (US); Gil Navon, Ramat Gan (IL); Alexej Jerschow, New York, NY (US)

(21) Appl. No.: 12/811,987

(22) PCT Filed: Jan. 7, 2009
(86) PCT No.: PCT/US09/30319

§ 371 Date: Sep. 28, 2010

Related U.S. Application Data
(60) Provisional application No. 61/019,439, filed on Jan. 7, 2008.

(51) Int. Cl. A61B 5/055

(52) U.S. Cl. 600/410

(57) ABSTRACT

An exemplary methodology, procedure, system, method and computer-accessible medium can be provided to determine one or more particular frequencies of cross-relaxation between at least one molecule and at least one particular compound, determine a chemical exchange based on magnetic resonance data using a further frequency which is different from the one or more particular frequencies, and derive particular information about the anatomical region of interest based on the chemical exchange.

### CROSS-REFERENCE TO RELATED APPLICATIONS

This application relates to and claims priority from U.S. patent application Ser. No. 61/019,439 filed Jan. 7, 2008, the entire disclosure of which is hereby incorporated herein by reference.

### STATEMENT REGARDING FEDERALLY-SPONSORED RESEARCH

The invention was developed in part with the U.S. Government support from the National Science Foundation under Grant Numbers CHE-0554400 and MRI-0116222, the National Institutes of Health under Grant Number 1R21AR054002-01A 1, RO1 AR 053133-01A2 and the National Center for Research Resources under Research Facilities Improvement Grant Number C06 RR-16572-01. Thus, the U.S. Government may have certain rights in the invention.

### FIELD OF THE INVENTION

The present invention relates to exemplary embodiments of a system, method and computer-accessible medium for the measurement of Glycosaminogycan (“GAG”) concentration, and further, for measuring a localized GAG concentration in vivo.

### BACKGROUND INFORMATION

Glycosaminogycans (“GAGs”) are involved in numerous vital functions in the human body. Mapping the GAG concentration in vivo may be desirable for the diagnosis and monitoring of a number of musculoskeletal diseases such as osteoarthritis (“OA”) and intervertebral disc (“IVD”) degeneration induced lower back pain, both of which affect millions of individuals.

GAG can include long unbranched carbohydrates with repeating disaccharide units, and may be classified into six categories: chondroitin sulfate-4 (“CS-4”), dermatan sulfate (“DS”), keratan sulfate (“KS”), heparin, heparan sulfate, and hyanluronan (“HA”) as described, e.g., in Dudhia, J. (2005) Cell. Mol. Life Sci. 62, 2241-2256. GAGs can play an important role, e.g., in human musculoskeletal function, cell regulation, and spinal function.

Some GAGs may function independently. Heparin, for example, can act as an anticoagulant in blood clotting, and may be used in anti-coagulation therapies. Other GAGs can form functional conglomerates with proteins and/or DNA. Mucopolysaccharidosis, a genetically inherited disease, can result from defects in lysosomal enzymes responsible for the metabolism of membrane protein-bound GAGs as described, e.g., in Knudson, A. G. et al. (1971) Proc. Natl. Acad. Sci. USA 68, 1738-1741. Proteoglycans (“PG”s), which can include a core protein and one or more covalently attached GAG chains, may play vital functions in diarthrodial joints as described, e.g., in Roughley, P. J. (2006) Eur. Cell Mater. 12, 92-101, and IVD as described, e.g., in Blumenkrantz, G. et al. (2006) Magn. Reson. Imaging 24, 1001-1007.

Osteoarthristis (OA), which may affect about 10% of the U.S. population (similar numbers are found for other developed countries), can be characterized by a loss of PGs in cartilage as described, e.g., in Moskowitz, R. W. et al. (2007) Osteoarthritis: Diagnosis and Medical/Surgical Management (Lippincott Williams & Wilkins, Philadelphia, Pa.). Thus, quantification of GAG concentration in vivo can be important for the understanding of the pathophysiology of many common diseases.

Currently, there isn't an applicable direct GAG mapping method available. A delayed gadolinium enhanced MRI contrast (“dGEMRIC”) technique can allow one to measure the GAG concentration indirectly in cartilage. In this technique, after intravenous administration of Gd(DTPA)2− diffuses into cartilage, the GAG concentration is roughly inversely proportional to the concentration of Gd(DTPA)2− due to charge repulsion. A negative charge density of cartilage tissue (and hence GAG concentration) can be inferred by monitoring the differences in the T₁ values with and without the agent as described, e.g., in the Moskowitz publication and in Burstein, D. et al. (2000) Invest. Radiol. 35, 622-638. Such technique, however, may not be useful for the analysis of IVDs and heart valves because of the slow diffusion of Gd(DTPA)²⁻ into the tissue (Niinimaki J L, Parviainen O, Ruohonen J, Ojala R O, Kurunlahti M, Karppinen J, Tervonen O, Nieminen M T. (2006). J Magn Reson Imaging 24, 796-800).

T_{1ρ} MRI can map GAG concentration in both diarthrodial joints and IVDs as described, e.g., in the Blumenkrantz publication and in Regatte, R. R. et al. (2003) Radiology 229, 269-274. The long time needed for the imaging by the technique limits its clinical applicability. The long preparation of T_{1ρ} magnetization by a long spin-lock pulse may impair its effectiveness in certain applications due to a high specific absorption rate (“SAR”) as described, e.g., in Wheaton Andrew, J. et al. (2004) Magn. Reson. Med. 51, 1096-102. With the advent of high field MRI scanners, ²³Na MRI can be used to analyze a concentration of positively charged ²³Na in cartilage. Such concentration may further be used to map negatively charged GAGs as described, e.g., in Lesperance, L. M. et al. (1992) J. Orthop. Res. 10, 1-13, and in Shapiro, E. M. et al. (2002) Magn. Reson. Med. 47, 284-291. Quadrupolar coupling of ²³Na in cartilage may also correlate with degeneration as described, e.g., in Ling, W. et al. (2006) Magn. Reson. Med. 56, 1151-1155, and in Shinar, H. et al. (2006) NMR Biomed. 19, 877-893. Such effect may be used in combination with quadrupolar contrast techniques to elucidate degeneration processes as described, e.g., in Ling, W. et al. (2005) J. Magn. Reson. 176, 234-238, in Choy, J. et al. (2006) J. Magn. Reson. 180, 105-109, in Ling, W. et al. (2006) Solid State NMR 29, 227-231, and in Eliav, U. et al. (2003) J. Magn. Reson. 165, 276-281. Clinical application of such techniques may be limited by the low signal-to-noise ratio, low sensitivity compared to proton and the need for special RF hardware.

Chemical exchange-dependent saturation transfer (“CEST”) is a technique which can be used to detect contrast in MRI procedures as described, e.g., in Guivel-Scharen, V. et al. (1998) J. Magn. Reson. 133, 36-45, and in Ward, K. M. et al. (2000) J. Magn. Reson. 143, 79-87. Exchangeable proton spins can be saturated when using the CEST technique, and the saturation can be transferred upon chemical exchange to the bulk water pool. As a result, a large contrast enhancement in bulk water can be achieved, which may reach a factor of about 10²-10⁶ for certain polymeric systems (relative to the concentration of the macromolecules) as described, e.g., in Zhou, J. et al. (2006) Prog. NMR Spectr. 48, 109-136. The CEST technique can been employed to image tissue pH as described, e.g., in Zhou, J. et al. (2003) Nature Med. 9, 1085-1090, to map brain proteins using—NH residues as described, e.g., in Zhou, J. et al. (2003) Magn. Reson. Med. 50, 1120-1126, to monitor glycogen concentration in liver as described, e.g., in van Zijl, P. C. M. et al. (2007) Proc. Natl. Acad. Sci. USA 104, 4359-4364, and to map the specific gene expression in vivo as described, e.g., in Gilad, A. A. et al. (2007) Nature Biotech. 25, 217-219.

Cartilage related diseases such as arthritis and lower back pain is also becoming a large problem worldwide. Structurally, the spine is a complex structure with alternating vertebrae and intervertebral discs, which are the largest avascular, aneural structures in the body, each unit consisting of two anatomical regions: the annulus fibrosus and the nucleus pulposus. Loss of GAG in the nucleus is the most marked degenerative change observed, and distribution of GAG in the nucleus is also of functional importance to define the swelling pressure. Between the vertebra and the disc, connecting the two, is the cartilaginous end plate, which consists of hyaline cartilage with a calcified layer close to the vertebra. Collagen represents about 15-20% of the nucleus and 65-70% of annulus dry weight whereas proteoglycan (PG) represents approximately 50% of the nucleus and 10-20% of annulus dry weight. The functional part of PG is also the negatively charged GAGs anchored on the core protein.

The onset and development of disc degeneration is likely to be multifactorial, including genetics, blocking of nutrition supply/regulating molecules, high frequency of overloading, or acute injury. All the factors above induce the aberrant cell activities or cell death, which disturbs the maintenance of the matrix. As a result, slow loss of PG in the nucleus occurs. The loss of PG leads to a reduction of the osmotic pressure and hence to a loss of hydration, thus significantly weakening the strength of the disc. Finally the shrinkage of the disc height is observed. It should be noted that the time course of disc degeneration is rather long, typically over decades. Thus, the diagnostics of early degenerative changes at the stage of cellular aberrance or macromolecular alteration in a structurally sound disc is vital for preventing the disease from compromising life quality since the chronic accumulation of the morphological change is hard to restore. Moreover, should such diagnostic techniques be available, they would be powerful tools to promote the clinical intervention.

### SUMMARY OF THE INVENTION

The above described problems can be addressed by exemplary embodiments of the system, method and computer accessible medium according to the present invention. For example, using such exemplary embodiments, it is possible to determine one or more particular frequencies of cross-relaxation between at least one molecule and at least one particular compound, determine a chemical exchange based on magnetic resonance data using a further frequency which is different from the one or more particular frequencies, and derive particular information about an anatomical region of interest based on the chemical exchange.

The magnetic resonance data can pertain to particular nuclei, where the particular nuclei can be exchangeable nuclei. The exchangeable nuclei can be protons. The exchangeable protons can be one of hydroxyl protons or amide protons.

The chemical exchange can be determined by measuring at least one of a chemical transfer or a magnetization transfer of the at least one molecule. The chemical transfer can be determined using a chemical exchange saturation transfer pulse sequence applied to target the at least one molecule. The particular information can be a concentration or a density of the at least one molecule.

The anatomical region of interest is at least one of a knee, an intervertebral disc, a cornea, a cervix or a heart valve. The derivation procedure can relate to at least one of osteoarthritis or IVD degeneration. The chemical exchange can be determined using an offset relative to a water signal of the at least one compound. The offset can be at least one of region around −2.6 ppm, −1 ppm, +1 ppm, +2.6 ppm or +3.2 ppm.

The nuclei can be isotopes of at least one of phosphorus, xenon, carbon, sodium or fluorine. The concentration or the density can be associated with at least one of glycosaminoglycan or proteoglycan.

An image can be generated of the anatomical region of interest. The image can be a difference image. The difference image can be an image showing a difference between two images relating to equal and opposite offsets. The at least one molecule can be a macromolecule.

Using such exemplary embodiments, it is also possible to determine a frequency region in which chemical exchange and cross-relaxation do not interfere.

Using such exemplary embodiments, it is also possible to select one or more particular frequencies of a cross-relaxation between at least one macromolecule and at least one particular compound, determine at least one of a chemical transfer or a magnetization transfer from magnetic resonance data using at least one of the particular frequencies, and derive particular information about the anatomical region of interest from at least one of the chemical transfer or the magnetization transfer.

These and other objects, features and advantages of the present invention will become apparent upon reading the following detailed description of embodiments of the invention, when taken in conjunction with the appended claims.

### BRIEF DESCRIPTION OF THE DRAWINGS

The foregoing and other exemplary objects of the present invention will be apparent upon consideration of the following detailed description, taken in conjunction with the accompanying exemplary drawings and claims, in which like reference characters refer to like parts throughout, and in which:

FIG. 1a is an illustration of an exemplary graph of a Z-spectrum of GAG in H₂O, together with a corresponding ¹H spectrum;

FIG. 1b is an illustration of an exemplary graph of a Z-spectrum of GAG in D₂O, together with a corresponding ¹H spectrum;

FIG. 1c is an illustration of an exemplary graph of a Z-spectrum of cartilage, together with a corresponding ¹H spectrum;

FIG. 1d is an illustration of an exemplary graph of a Z-spectrum of cartilage equilibrated in D₂O, together with a corresponding ¹H spectrum;

FIG. 2a is an illustration of an exemplary graph of Z-spectra of a cartilage trysinization series with asymmetry values shown in both the amide and hydroxyl region;

FIG. 2b is an illustration of an exemplary graph of —OH hydroxyl proton CEST vs. [²³Na], as assessed using Na NMR spectroscopy;

FIG. 2c is an illustration of an exemplary graph of —NH hydroxyl proton CEST vs. [²³Na], as assessed using Na NMR spectroscopy;

FIG. 3a is an illustration of an exemplary series of CEST images taken from a trypsinization series on a bovine patella;

FIG. 3b is an illustration of an exemplary graph of CEST contrast extracted according to Eq. (2) herein;

FIG. 4a is an illustration of an exemplary series of in vivo images of a human patella using irradiation at δ=−1.0 ppm, δ=+1.0 ppm, and a corresponding difference image;

FIG. 4b is an illustration of an exemplary graph of extracted CEST contrast for a femur, and for the lateral and medial sides of a patella;

FIGS. 5a and 5b are illustrations of exemplary graphs of a Z-spectra of a nucleus in an intact state and a depleted state;

FIG. 6a is an illustration of an exemplary image of a CEST of a nucleus of an intervertebral disc in an intact and depleted state;

FIG. 6b is an illustration of an exemplary ²³Na image of the nucleus of an intervertebral disc in an intact and depleted state on the same sample of FIG. 6a;

FIG. 6c is an illustration of an exemplary image of a CEST of a nucleus of an intervertebral disc in an intact and depleted state;

FIG. 6d is an illustration of an exemplary ²³Na image of the nucleus of an intervertebral disc in an intact and depleted state on the same sample of FIG. 6c;

FIGS. 7a-7f are illustrations of exemplary Porince disc images in vitro.

FIG. 8 illustrates a flow diagram according to another exemplary method of the present invention; and

FIG. 9 illustrates a block diagram of an exemplary embodiment of a system according to the present invention.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention.

### DETAILED DESCRIPTION OF EXEMPLARY EMBODIMENTS OF THE INVENTION

Exemplary embodiments of the methodology and procedure which can be implemented by the exemplary system, method and computer-accessible medium according to the present invention will now be described with reference to the figures.

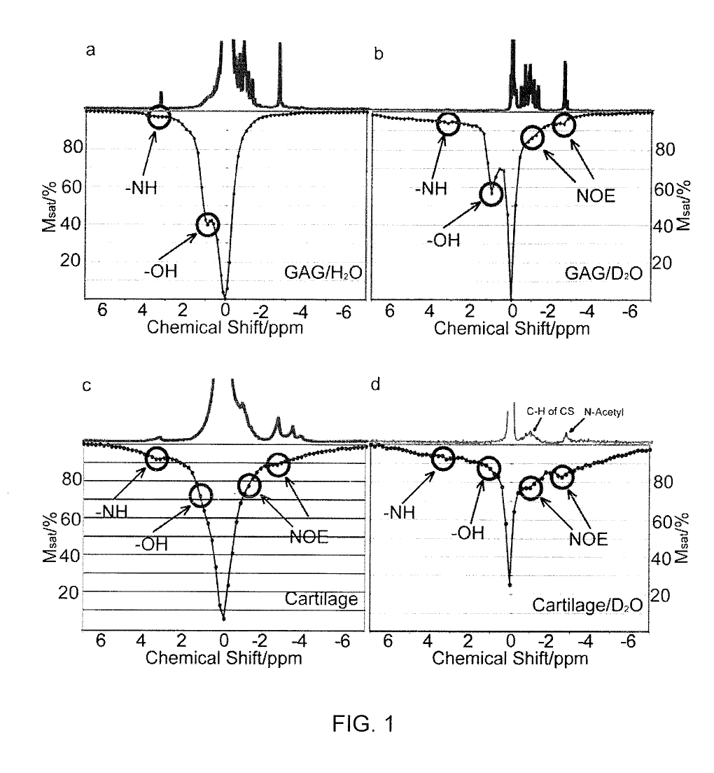

FIG. 1a shows an exemplary z-spectrum of a GAG phantom, as described, e.g., in Hinton, D. P. et al. (1996) Magn. Reson. Med. 35, 497-505. In this exemplary figure, labile proton sites at δ=+3.2 and +1.0 ppm downfield from the water signal are shown. FIG. 1c shows an exemplary z-spectrum from a piece of bovine cartilage which appears more complex than that of the GAG phantom shown in FIG. 1a. Such complexity may arise, e.g., from additional magnetization transfer mechanisms that can stem from a macromolecular nature of the assembly.

In addition to the exemplary two labile sites downfield of water described above, two sites at δ=−2.6 ppm and −1.0 ppm upfield of water can also be identified. These two exemplary sites can correspond to CH and N-acetyl residues, respectively, in GAG as described, e.g., in the Ling 2007 Publication and in Schiller, J., Naji, L., Huster, D., Kaufmann, J. & Arnold, K. (2001) MAGMA 13S 19-27. The protons in these residues may not be exchangeable, and their appearance in the z-spectrum may thus be related to a magnetization transfer mechanism other than a chemical exchange. Such a mechanism can be, e.g., a nuclear Overhauser effect—saturation transfer (“NOEST”). An exemplary NOE enhancement of a water signal can be expressed as:

NOE_{water}=T₁·σ, (1)

where T₁ represents the longitudinal relaxation time of water, and a represents a cross-relaxation constant between water and GAG, as described, e.g., in Noggle, J. H. & Schirmer, R. E. (1971) The nuclear Overhauser effect, chemical application (Academic Press, New York, N.Y.). σ can be negative when ω₀τ_{c}≧1, where ω₀ can represent a Larmor frequency and τ_{c} can represent a rotational correlation time.

A negative cross-relaxation between water and the macromolecule signal for glycogen is described, e.g., in Chen, W. et al. (1993) Biochemistry 32, 11483-7, where a negative NOE was observed for the glycogen protons upon irradiation of the water protons. FIGS. 1b and 1d suggest a presence of a NOE based on the observation that NOE and chemical exchange respond differently to the presence of bulk D₂O. FIG. 1b shows an exemplary z-spectrum of GAG in 95% D₂O, and FIG. 1d shows an exemplary z-spectrum of cartilage equilibrated in D₂O. For example, although the two upfield sites and the two downfield sites can demonstrate a similar behavior in the z-spectra in the presence of bulk water, they can be observed to behave differently in the presence of bulk D₂O. For example, the presence of bulk D₂O can significantly reduce an appearance of dips at the two chemical exchangeable sites (δ=+3.2 ppm and +1.0 ppm), while it may enhance a NOE at δ=−1.0 ppm and −2.6 ppm, as shown in FIGS. 1b and 1d. Such exemplary enhancement may be attributed in part to an increase of T1 in bulk D₂O as compared to that in bulk H₂O, similar to that described, e.g., in Chen, W. et al. (1993) Biochemistry 32, 9417-22.

T1 value of the water protons was observed to be 4.3 s and 9.7 s for GAG solutions in H₂O and in 90% D₂O respectively, and it was observed to be 2.1 s and 4.0 s in native cartilage and in D₂O-equilibrated cartilage, respectively. Thus, the NOE from the CH and N-acetyl groups of GAG can lead to a magnetization transfer mechanism which may be visible at the two sites at δ=−1.0 ppm and −2.6 ppm upfield of water.

The dips of the peaks at δ=−1.0 ppm and −2.6 ppm are due to non-exchangeable protons and therefore do not result from CEST but rather from NOEST. Since, e.g., upon replacing the exemplary bulk H₂O solution with 90% D₂O, the concentrations of the non-exchangeable protons stays the same, their appearance in the z-spectra is more pronounced. By contrast, the exchangeable groups show a reduced intensity in the z-spectra, since they are replaced by deuterium as shown in FIGS. 1b and 1d. Thus, the NOEST from the CH and N-acetyl groups of GAG can lead to a magnetization transfer mechanism which may be visible at the two sites at δ=−1.0 ppm and −2.6 ppm upfield of water.

In an exemplary procedure according to an exemplary embodiment of the present invention, to examine the applicability of CEST/NOEST for clinical diagnosis of OA, z-spectra of a cartilage trypsinization series were acquired. Trypsin may primarily act on proteoglycan (“PG”) and can reduce a GAG concentration as described, e.g., in Bartholomew, J. S. et al. (1985) Biochem. J 227, 429-37. Corresponding ²³Na concentrations (“[²³Na]”) can be recorded using ²³Na NMR. [²³Na] was observed to decrease as a result of PG depletion, and can be used as an indicator of GAG concentration as described, e.g., in the Lesperance publication and in the Shapiro publication.

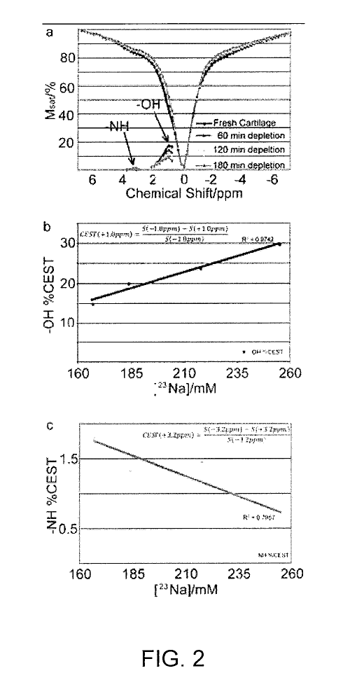

FIG. 2s shows an exemplary z-spectra acquired from a piece of fresh cartilage, and from the same piece of cartilage after sequential trypsinization. Asymmetry plots are shown along with the z-spectra in the exemplary graph of FIG. 2a. Such exemplary asymmetry plots represent a difference between the upfield side and the downfield side of the plot, and may be expressed as:

MTR_{asym}(δ)=[S(−δ)−S(+δ)]/S₀, (2)

where δ can represent an offset of the irradiation relative to the water signal, and S(δ) and S₀ can represent water intensities after a long saturation pulse at offset δ and at a large offset (+40 ppm), respectively, as described, e.g., in the Zhou (2003) publication.

Another relationship described, e.g., in the Ward publication, may express the CEST effect more intuitively as:

CEST(δ)=[S(−δ)−S(+δ)]/S(−δ), (3)

where δ can represent an offset of the irradiation relative to the water signal, and S(δ) can represent a water intensity acquired after a long saturation pulse at an offset δ.

FIG. 2b shows an exemplary relationship between CEST(δ=+1.0 ppm) and [²³Na] in accordance with Eq. (3) herein. The substantially linear relationship observed between CEST(δ=+1.0 ppm) and [²³Na] suggests that the —OH hydroxyl proton can be used as a gagCEST agent.

As shown in the exemplary graph of FIG. 2c, the —NH amide proton CEST(δ6=+3.2 ppm) shows a negative correlation with [²³Na] which is due to the NOEST effect at δ=−3.2 ppm. Thus, this can indicate that the NOE effect can even override certain exchange effects in intensity.

The smaller —NH CEST effect can result from a lower chemical exchange rate of the amide protons as compared with that of the hydroxyl protons. The exchange rates can be estimated based on a measured CEST value. Such estimation may be accurate under an assumption of complete saturation of the irradiated group as described, e.g., in the Zhou 2006 publication, which may be expressed as:

k_{sw}=(M_{0w}/M₀ₛ)·[CEST/(1−CEST)]·R_{1w}, (4)

where k_{sw} can represent an exchange rate of protons from —NH or —OH to water, R_{1w} can represent a reciprocal of water T1, and M₀ₛ, M_{0w} can represent equilibrium magnetizations of labile protons and water protons, respectively.

The —OH and —NH concentrations in the 125 mM GAG phantom are 375 mM and 125 mM, respectively. The exemplary observed CEST effect was 95.2% for —OH and 8.9% for —NH, and water T1 in the phantom was observed to be about 4.3 s. These values suggest that k_{—NH}≈20 s⁻¹ and k_{—OH}≈1350 s⁻¹ based on Eq. (4) herein, as described, e.g., in Koskela, H. et al. (2007) J. Biomol NMR. 37, 313-320, and in Liepinsh, E. et al. (1996) Magn. Reson. Med. 35, 30-42.

Thus, a high concentration and high chemical exchange rate of —OH can override NOE signals at δ=−1.0 ppm, and can lead to a substantial CEST effect (e.g., about 15%˜30%). The exemplary graph in FIG. 2b exhibits a linear dependence of this effect on the GAG concentration, which further demonstrates that —OH hydroxyl protons may be used as gagCEST agents.

The exemplary embodiment of the cartilage/GAG system thus allows us to demonstrate (1) avoiding the NOE allows one to observe enhanced CEST effects (FIG. 2b), and (2) using NOE allows one to obtain enhanced contrast when exchange effects are weak or neglectible, or when a large fraction of solvent molecules are not NMR-active (as in the case of 90% D₂O in FIGS. 1b, 1d).

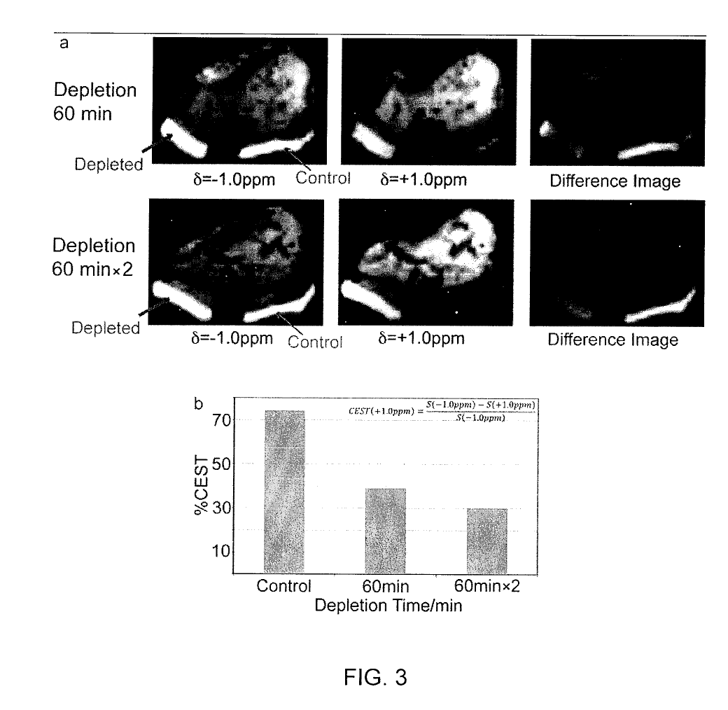

FIG. 3a shows exemplary images of an ex vivo CEST imaging application of a bovine patellar cartilage sample at δ=+1.0 ppm. The cartilage on the surface of the patella was divided into a control and a PG-depleted region. The control region was left intact, while the depleted side was typsinized twice for 60 min, with an imaging scan performed after each typsinization procedure. A decrease of signal intensity in the trypsinized region can be seen in the exemplary images shown in FIG. 3a. The difference image represents the subtraction of the image at δ=+1.0 ppm from the image at δ=−1.0 ppm.

An extracted graph of gagCEST effect vs. depletion time is shown in the exemplary graph of FIG. 3b. The data values shown in this graph suggest that gagCEST techniques can be utilized for the assessment of GAG using MRI techniques.

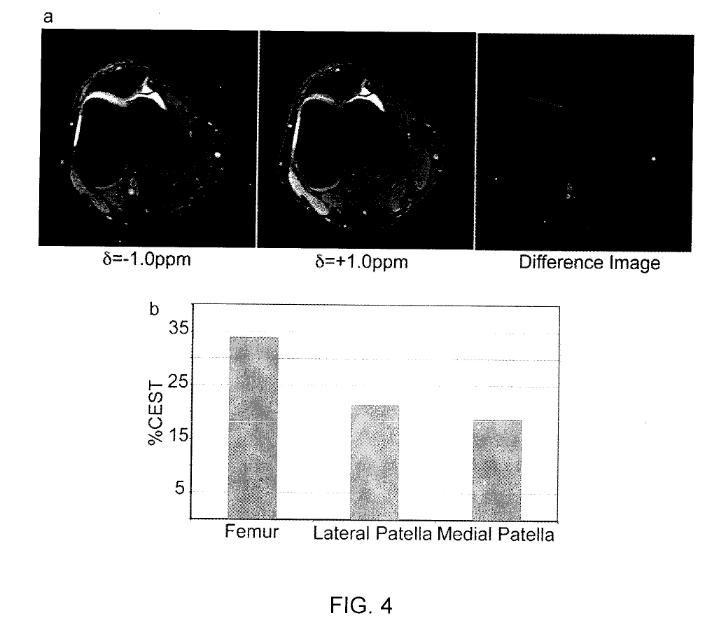

FIG. 4a shows exemplary images of an in-vivo analysis of a patellofemoral human knee joint using an —OH CEST technique. These exemplary images reveal a clear demarcation of a cartilage lesion on the medial facet. The accumulation of joint effusion (e.g., fluid) in the knee (which can appear in such images as a brightness in the diarthrodial joint) is almost totally removed in the exemplary difference image shown in FIG. 4a. In the difference image, a loss of GAG concentration can be observed on the medial side of the patellofemoral knee joint. The regional variation of the GAG concentration can be observed in the knee joint across the cartilage (˜33%), the lateral side of patella (˜22%) and the medial side of the patella (˜18%), as shown in FIG. 4b. These results appear consistent with the ex vivo analysis (˜30%) shown in FIG. 2b. Further, regional variation of GAG concentration described herein appears consistent with a previous analysis performed using sodium MRI and described, e.g., in Shapiro, E. M. et al. (2002) Magn. Reson. Med. 47, 284-291.

Thus, a CEST difference image can be utilized to detect localized GAG concentration distributions and hence a pathological state of cartilage in vivo. For example, GAG —OH sites have been observed at δ=+0.9-+2.6 ppm (relative to the water resonance). Such —OH sites may provide an endogenous CEST agent which can be used for assessing GAG concentration in vivo. A high concentration of such sites (e.g., 200 mM˜300 mM) along with their fast exchange rates (e.g., ˜103 Hz) can produce a large CEST effect at relatively low saturation power (e.g., <100 Hz in tissue) and with short presaturation time. Such properties can allow measurements to be made with a lower specific absorption rate (“SAR”) as compared to other conventional techniques such as, e.g., Tlp.

Utilization of —NH amide protons in CEST analyses at δ=+3.2 ppm (which is equivalent to NOEST at δ=−3.2 ppm) can provide certain advantages. For example, a larger irradiation offset may permit application of a higher saturation power, and fewer artifacts may be caused by direct water saturation.

Cartilage may be a useful tissue type for demonstrating application of a gagCEST/gagNOEST method because of the medical significance of OA. However, such method can be used to analyze a variety of tissues containing GAG or other compounds. For example, degeneration of an IVD, which may be associated with disc degeneration disease, can lead to low back pain. In IVD tissue, a nucleus pulposus containing about 50% PG (by dry weight) can impart a compressive stiffness on an IVD, and thus on a human trunk as described, e.g., in the Blumenkrantz publication cited herein. A dGEMRIC technique may not be applicable to IVDs as described, e.g., in Hwang, G. J. et al. (1997) J. Magn. Reson. Imaging 7, 575-578. For example, uptake of Gd(DTPA)²⁻ may cause unpredictable side effects on spinal neurons nearby. The feasibility of T_{1ρ} MRI techniques for analyzing IVD tissue has been described, e.g., in the Blumenkrantz publication cited herein. However, certain characteristics of a gagCEST/gagNOEST method may be preferable for MR imaging of IVD. For example, a gagCEST/gagNOEST method can use less power and may provide faster data acquisition rates that the other techniques described herein.

Further, GAG concentration via Chemical Exchange Saturation Transfer (“gagCEST”) has been found to be helpful in analyzing various data relating to interverbral disc and degenerative disc disease. GagCEST has been found to map GAG concentration and GAG distribution in the disc, and to non-invasively measure the pH value in discs.

Disc degeneration development occurs in three relative stages. The first stage is when an aberrance of cell metabolism occurs, which can be detectable after days or months. In the second stage, there is an alteration of matrix composition of the disc, which is detectable after years or decades. In the third stage, there is a morphological change in the disc. GagCEST may apply to a pathologic state (but not limited to), such as arthritis and back pain, both in the early stage evidence-based disease prevention/interventions and in post-treatment evaluation, e.g. monitoring cartilage resurfacing longitudinally or growth factor treatment in lower back pain patients.

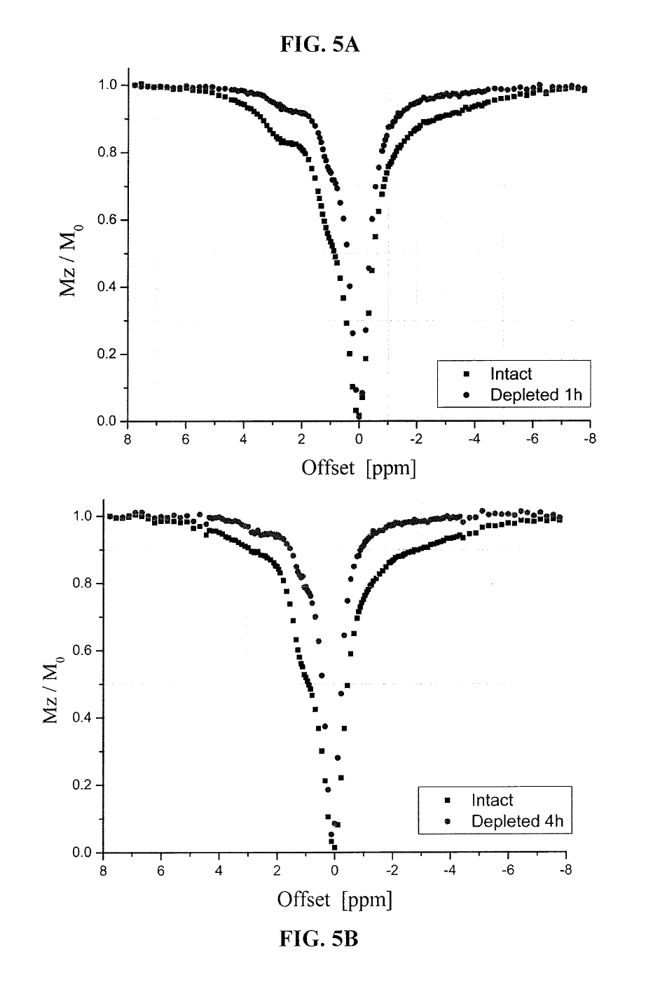

As shown in the exemplary graphs of FIGS. 5a and 5b, the preliminary z-sectra of a nucleus in an intact and depleted state shows that hydroxyl protons CEST can be used to evaluate GAG concentration and distribution of a porcine disc in vitro. FIG. 5a illustrates an exemplary graph of a z-spectrum of an intact nucleus, vs. a z-spectrum of a depleted nucleus after one hour. FIG. 5b illustrates an exemplary graph of a z-spectrum of an intact nucleus, vs. a z-spectrum of a depleted nucleus after four hours.

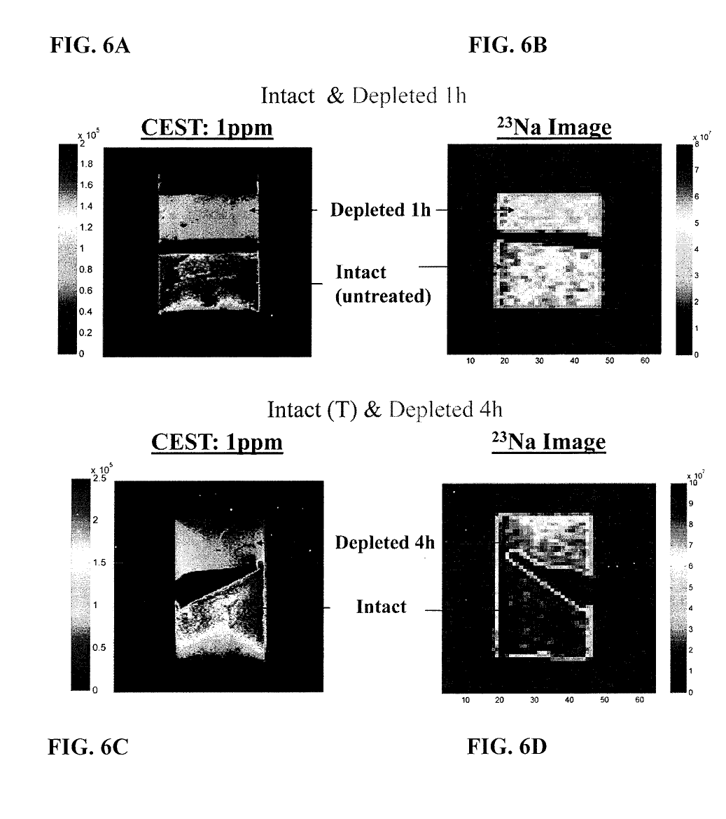

The CEST images at 1.0 ppm are then obtained on a disc nucleus of porcine spine after 1 hour and 4 hours of trypsin treatment, respectively. As shown in the exemplary image of FIG. 6a, after 1 hour of depletion, a CEST image has a 40% of intensity reduction, and its corresponding exemplary ²³Na image in FIG. 6b has an approximately 13% reduction. As shown in the exemplary image of FIG. 6c, after 4 hours of depletion, a CEST image has a 52% of intensity reduction and its corresponding ²³Na image has an approximately 18% reduction. The high concentration of GAG in the nucleus is the reason of high sensitivity of the CEST contrast.

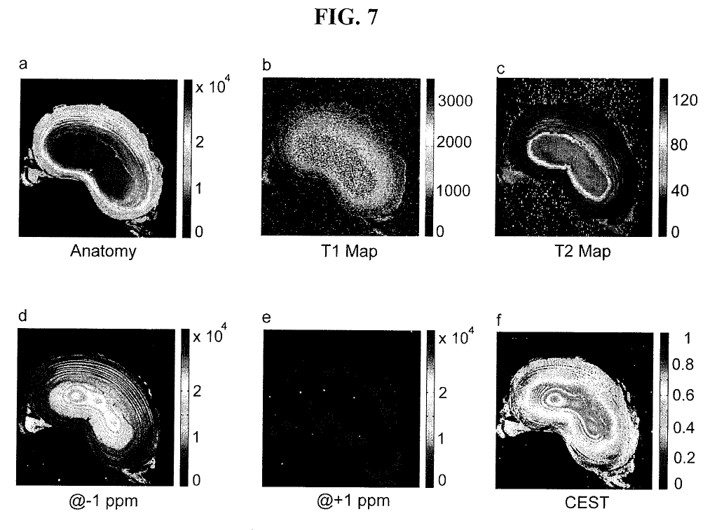

This sensitive contrast is further demonstrated in the exemplary images of FIG. 7 of a porcine disc. FIGS. 7a, 7b and 7c illustrate exemplary axial images of an ex vivo intact porcine disc assessed by routine MRI techniques. FIGS. 7d, 7e and 7f illustrate exemplary images using gagCEST to evaluate GAG concentration on the same disc, including an exemplary image at −1.0 ppm image (FIG. 7d), an exemplary image at +1.0 ppm (FIG. 7e), and an exemplary CEST image (FIG. 7f). gagCEST demonstrates robust contrast in both the annulus and nucleus regions, and the nucleus region is shown much stronger than that from the annulus region. Moreover, although the anatomical image in FIG. 7a shows structural differences in AF and NP, little information is provided about the GAG distribution. The GAG concentration and its distribution is of critical importance to disc function and physiology. From the results, it can be demonstrated that measurement of GAG concentration and distribution on a human disc in vivo is feasible.

Further, pH imaging may also be helpful in the prevention of IVD degeneration. It has been shown that amide proton based CEST effect could produce pH sensitive contrast in MRI, and that this pH sensitive MRI contrast can be used to evaluate ischemic rat brain although amide proton concentration was about millimolar range. The chemical exchange rates of both amide and hydroxyl proton are sensitive to a pH of approximately 5.6-7.6, which is within the physiological pH range in the extracelluar matrix of the nucleus. Additionally, the GAG concentration in the nucleus is high, up to 50% by dry weight, which gives rise to approximately 200-300 mM GAG concentration in a healthy nucleus. As a result, the amide proton concentration in the nucleus is approximately 200-300 mM. Concentration of hydroxyl proton is 3 times higher than that of amide proton. Moreover, collagen (15-20% by dry weight) and non-collagenous proteins also contribute substantial amide proton in the nucleus. Consequently, the high concentration of exchangeable protons in the nucleus offers high sensitivity for MRI pH contrast per pH unit. From these facts, it can be determined that amide/hydroxyl proton CEST based pH imaging in the nucleus is also feasible.

Other tissues or cell clusters which contain a considerable GAG concentration such as, e.g., heart valves, corneas and cervices, can also be analyzed using a gagCEST/gagNOEST method together with an appropriate imaging sequence. For example, as described herein, a negative NOE can result from an interaction between water and non-exchangeable GAG protons having a low mobility such that, e.g., ω₀τ_{c}≧1. This value for PG in cartilage as measured by ¹³C spectroscopy was reported to be ˜50 ns as described, e.g., in Torchia, D. A. et al. (1977) J. Biol. Chem. 252, 3617-25. Thus, ω₀τ_{c} may be much greater than 1 for proteoglycan in cartilage tissue. Therefore, even if a correlation time for a GAG proton-water proton dipolar-dipolar interaction is shorter than that based on ¹³C NMR spectroscopy, the relationship ω₀τ_{c}≧1 may likely be satisfied at lower magnetic fields such as, e.g., a 1.5 T field which may be used in a clinical scanner. The NOE from GAG can therefore provide a significant contribution to a CEST process, which may be detected by a clinical scanner.

Further, the size of the NOE peak relative to that of the water peak can be considerably larger for intact cartilage as compared to that from the GAG phantom in z-spectroscopy (c.f. FIGS. 1b and 1d). This difference can indicate a more restricted motion of GAG in cartilage, because it can be anchored on collagen fibrils. Such restriction may be consistent with similar observations obtained using ¹³C NMR spectroscopy as described, e.g., in the Torchia publication.

NOE may likely contribute to a low efficiency of further CEST based applications in vivo as described, e.g., in the Zhou (2006) publication, unless it is carefully avoided by choosing different irradiation frequencies, irradiation power and duration modifications. NOEST is still applicable even if there are no chemically exchangeable sites, where their resonance frequencies coincide with or are too close to the water resonance, or when the exchange sites are unusable due to too short T₁ and too slow exchange processes. Furthermore, the NOE sites are not exchanged by NMR inactive nuclei (as in the 90% D₂O example in FIGS. 1b, 1d, where the NOE sites actually become more pronounced than the CEST sites). As shown in FIG. 2c, the NOEST effect may also override the CEST effect, and hence become the primary mechanism for transfer and the enhancement of contrast. Pulse sequences may also be designed which enhance either NOEST or CEST.

### NMR Example

Exemplary NMR measurements in accordance with exemplary embodiments of the present invention were performed. For example, a sample containing a GAG concentration of 125 mM was prepared from CS A (Aldrich-Sigma, St. Louis, Mo., USA) in a standard phosphate buffered saline (“PBS,” pH=7.4, cell culture, Aldrich-Sigma). A further sample containing a GAG concentration of 125 mM was prepared from CS A in PBS/95% D₂O solution (D₂O, Aldrich-Sigma). These concentrations are based on a number of disaccharide units present in GAG or other compounds.

Bovine cartilage samples (including those used in the exemplary MRI example described herein) were obtained from a USDA approved slaughterhouse (Bierig Bros, Vineland, N.J.) within five hours of animal sacrifice (4-6 months old cows) and frozen at −20° C. until used. After de-icing, the soft tissue was first removed. The cartilage samples were cut to include various anatomical regions of cartilage, without a bone segment. Samples were placed into a 5 mm NMR tube. Sample sizes were about 4 mm in diameter and 5 mm in length. Fluorinated oil (Fluorinert, FC-77, Aldrich-Sigma) was used to fill void spaces for protection and to reduce a presence of susceptibility artifacts.

Trypsinization was performed as follows: a cartilage sample was immersed in a trypsin/PBS bath (containing 0.2 mg/mL trypsin, Aldrich-Sigma) for 60 min, after which it was placed into PBS for another 30 min. This procedure was performed 3 times for each sample treated. In cartilage D₂O equilibration experiments, fresh cartilage was immersed in the PBS/D₂O solution for 24 hours before it was sealed with fluorinated oil. A sample of 137 mM ²³Na in a mixture of PBS/4% agarose gel was prepared to calibrate [²³Na] in fresh cartilage. In this sample, ²³Na can exhibit relaxation properties similar to those of fresh cartilage.

NMR data were acquired at a field strength of 11.7 T (500 MHz ¹H frequency) using a Bruker Avance spectrometer equipped with a BBO probe. The temperature of the sample was stabilized at about 310 K with a variation of about ±0.2 K.

NMR measurements based on ¹H spectroscopy were performed with a hard pulse power of ω₁/2π=2.3 kHz and a 5° pulse width. A spectral width of 10 kHz was used and 8000 data points were recorded. Eight transients were acquired for each spectrum using a repetition delay of 1 s. Measurement of water T₁ was performed using a saturation recovery technique as described, e.g., in Mao, X. A. et al. (1997) Concepts Magn. Reson. 9, 173-187. Three 90°-crusher gradient pairs were used before a readout 90° pulse to remove transverse bulk magnetization. The three crusher gradient pairs of 1 ms duration each were performed using field gradients of 0.05 T/m, 0.1 T/m and 0.15 T/m, respectively. The total time for each saturation recovery measurement was approximately 30 minutes, and the following 8 delays were used in all of the spin-lattice measurements: 20 s, 10 s, 5 s, 2 s, 1 s, 500 ms, 100 ms and 1 ms. Four transients were acquired for each delay using a repetition delay of 30 s.

²³Na single pulse experiments were performed with a 90° pulse having w ₁/27c=22 kHz and 64 transients. A 250 ms recycle delay and 10 kHz window width were used. Continuous wave irradiation (“CW”) was used for the CEST experiments, with irradiation power and duration varying according to the system of interest, followed by a 5° pulse. Eight accumulations were used with a window width of 10 kHz, 8000 data points were collected, and a recycle delay was set to 8 s.

A total of 71 z-spectra were collected with a 100 Hz shift in offset frequency per step. Water intensity was then plotted as a function of irradiation frequency with respect to the center of the main water resonance. To estimate a chemical exchange rate of GAG, the 125 mM GAG phantom was irradiated for 10 s at δ=+/−3.2 ppm and 20 s at δ=+/−1.0 ppm, with an irradiation power of 250 Hz. Saturation duration and power levels of the presaturation were as follows: FIGS. 1a and 1c—4 s and 100 Hz; FIG. 1b—50 s and 50 Hz; FIG. 1d—10 s and 50 Hz; FIG. 2: 4 s and 250 Hz. Results of the exemplary NMR studies are shown in these exemplary figures and are described herein above.

### MRI Example

Extraneous tissue (e.g., ligaments, fat, etc.) of a fresh bovine patella was removed, and a groove was made in the middle of the patella on the articular surface. The patellae were then placed in a chamber containing a nonpermeable divider such that the groove was wedged on the divider. The control side of the patella was equilibrated in 137 mM PBS and the depleted side was immersed in a fresh trypsin-bath for two periods of 60 min. each. After each depletion, the entire patella was equilibrated in a PBS buffer solution for 30 min. A wedge-shaped gap was formed as a marker on the control side of the patella for ease of identification in MRI scanning.

For IVD, the nucleus was selectively cut and treated before put into MRI scanner (FIG. 5 and FIG. 6). The whole IVD sample containing all anatomical regions was put into an MRI scanner without treatment (FIG. 7).

An in vivo MRI analysis was performed on the knee of a human subject in accordance with exemplary embodiments of the present invention. A right knee joint of one human subject (male, age 30 years) with occasional knee pain was under MR investigation. A comprehensive medical review of this subject is ongoing. The subject was asked to rest for at least 30 min. before the imaging session was conducted.

The MRI measurements were performed using a 3.0 T clinical MR scanner (Magnetom Tim Trio, Siemens Medical Solutions, Erlangen, Germany). An 18-cm diameter, 8 channel transmit-receive phased-array (“PA”) knee coil was utilized for all imaging measurements.

CEST imaging sequences were modified based on a spoil gradient GRE sequence using a train of ten 180° Gaussian pulses having a pulse length of 31 ms, an interval of 1 ms, an offset of 1.0 ppm, and an average saturation power of 35 Hz. Acquisition parameters utilized for the CEST image of the patella were: number of sections=5; TR/TE=2070/4 ms; section thickness=3 mm; acquisition matrix=256×128; FOV=150 mm×150 mm.

To meet the image load requirement, a 1 L phantom of 4% agarose was installed at the bottom of the patella samples. However, this rendered fat saturation on the patella inapplicable. The same saturation parameters were used on the human subject, except that the acquisition matrix was 256×256. Conventional selective fat suppression was used as described, e.g., in Rosen, B. R., Widen, V. J. & Brady, T. J. (1984) J. Comput. Assist. Tomogr. 8, 813-818.

The sodium concentration [²³Na] was calibrated based on a ²³Na signal intensity from the 137 mM 23Na/4% agarose system. By counting the volume factor of cartilage and 23Na/agarose in the NMR coil, [²³Na] was determined to be about 255 mM in fresh cartilage. The imaging processing was performed with ImageJ (an image processing utility described on the website http://rsb.info.nih.gov/ij).

In the exemplary graphs of FIGS. 3b and 4b, a CEST contrast was extracted by segmentation of a region of interest, measurement of mean signal intensities, and calculation of the CEST contrast in accordance with Eq. (3) herein.

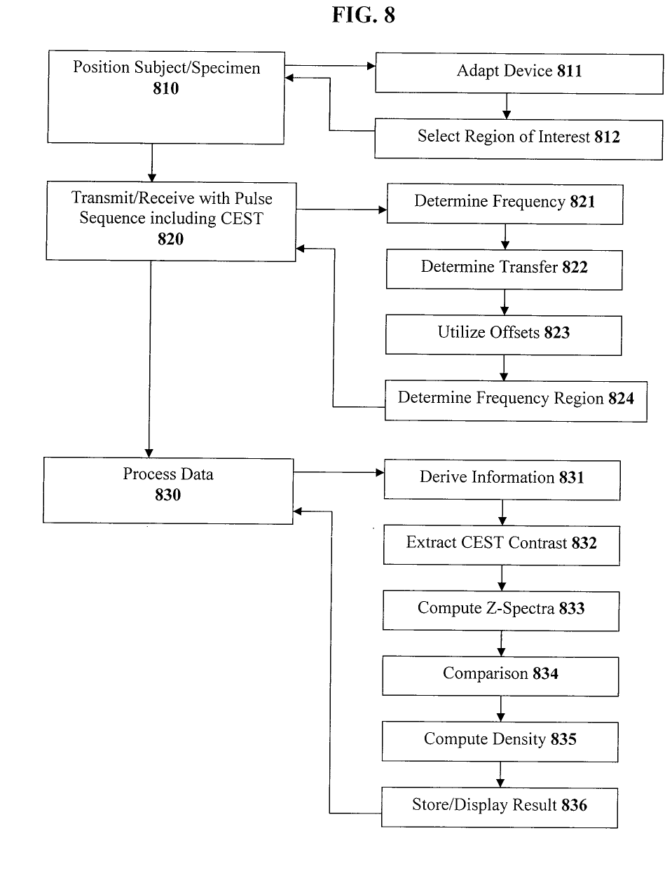

An exemplary flowchart in accordance with the present invention will now be described with reference to FIG. 8. First, a subject or a specimen is positioned relative to an imaging device at step 810. This step can include adapting a device at step 811 for at least one nuclei of at least one of phosphorus, xenon, carbon, sodium or fluorine. Next, at step 811, an anatomical region of interest can be selected of the subject/specimen, which can be at least one of a knee, a disc, a cornea of an eye or a heart valve, for example.

Then, the image can be transmitted/received using a pulse sequence including CEST at step 820. This step can include determining one or more particular frequencies of a cross-relaxation between at least one macromolecule and at least one particular compound, at step 821. Step 822 provides for determining at least one of a chemical transfer or a magnetization transfer from magnetic resonance data, using at least one of the particular frequencies determined at step 821 and pertaining to exchangeable nuclei, where the protons are preferably at least one of hydroxyl protons or amide protons. At step 823, one or more offsets can be utilized, e.g. −2.6 ppm, −1 ppm, +1 ppm, +1.9 ppm, +3.2 ppm, etc. At step 824, a frequency region can be determined in which chemical exchange and cross-relaxation do not interfere. Other parameters can also be provided for within step 820, e.g., illustrative parameters, parameters that vary, spoil gradient GRE, Gaussian pulses, pulse lengths, intervals, average saturation power, number of sections, TR/TE, section thickness, acquisition matrix, and FOV.

At step 830, the data is processed, which can include deriving particular information about the anatomical region of interest from at least one of the chemical transfer or the magnetization transfer preferably for at least one of glycosaminoglycan or proteoglycan, at step 831. The CEST contrast is extracted at step 832, which can include the segmentation of ROI, measurement of mean signal intensities, and the calculation of CEST contrast using Equation 3 (as described above). Then, at step 833, the z-spectra can be computed, and analyzed relative to e.g. —OH, —NH, etc. The results can be optionally compared to baseline or normative data at step 834 to produce a difference image. At step 835, the density and/or concentration of the molecule/macromolecule of interest is computed. The results can either be stored in a storage device or provided for display at step 836.

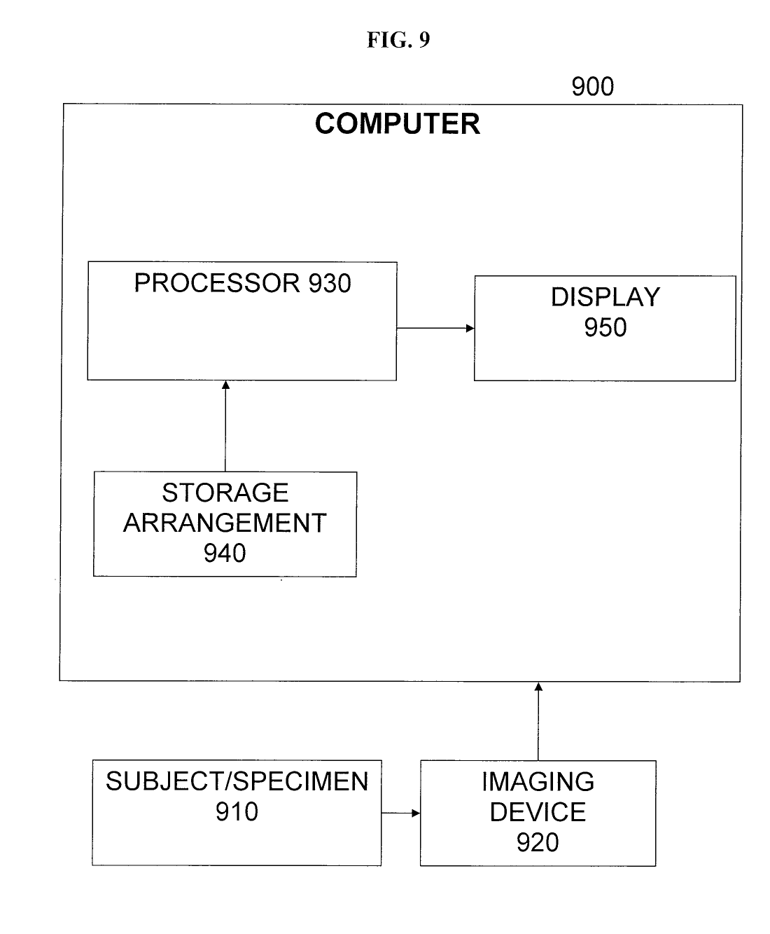

FIG. 9 illustrates a block diagram of an exemplary embodiment of a system according to the present invention. A computer 900 can be provided having a processor 930 which can be configured or programmed to perform the exemplary steps and/or procedures of the exemplary embodiments of the techniques described above. For example, a subject/specimen 910 can be positioned and an anatomical region of interest can be selected on the subject/specimen 910, as provided for in step 810 above. The imaging device 920 can be used to obtain images including CEST as provided for in step 820 above. The data/images can be provided from the imaging device to the computer 900, which can be transmitted to the processor 930 and/or storage arrangement 940.

According to one exemplary embodiment of the present invention, the data can be stored in a storage arrangement 940 (e.g., hard drive, memory device, such as RAM, ROM, memory stick, floppy drive, etc.). The processor 930 can access the storage arrangement 940 to execute a computer program or a set of instructions (stored on or in the storage arrangement 630) which perform the procedures according to the exemplary embodiments of the present invention. Thus, e.g., when the processor 930 performs such instructions and/or computer program, the processor 930 can be configured to perform the exemplary embodiments of the procedures according to the present invention, as described above herein.

For example, the processor can determine one or more particular frequencies of cross-relaxation between at least one molecule and at least one particular compound, determine a chemical exchange based on magnetic resonance data using a further frequency which is different from the one or more particular frequencies, and derive particular information about the anatomical region of interest based on the chemical exchange. This information can be received directly from the imaging device 920 or accessed from the storage arrangement 940. The processor 930 can then determine information for measuring GAG concentration, and further, measuring a localized GAG concentration in vivo.

A display 950 can also be provided for the exemplary system of FIG. 9. The storage arrangement 940 and the display 950 can be provided within the computer 900 or external from the computer 900. The information received by the processor 930 and the information determined by the processor 930, as well as the information stored on the storage arrangement 940 can be displayed on the display 950 in a user-readable format.

Exemplary embodiments of the system, method and computer accessible medium according to the present invention can make it possible to measure Glycosaminogycan (“GAG”) concentration, and further, measure a localized GAG concentration in vivo with high sensitivity by exploiting the exchangeable protons of GAGs, which can provide a powerful diagnostic MRI system and method.

Exemplary embodiments of the present invention can provide a method for GAG assessment, which can use labile protons residing on the GAGs. Such an exemplary method can allow the direct measurement of GAG concentrations in vivo. For example, a spectroscopic study of cartilage indicated that both amide protons (−NH, δ=+3.2 ppm downfield from the water signal) and hydroxyl protons (—OH δ=+1.0 to +1.9 ppm downfield from the water signal) within GAG molecules may be suitable for use as CEST agents as described, e.g., in Ling, W. et al. (2007) NMR Biomed. 20, 555-561. For example, a GAG unit can include one —NH amide proton and three —OH hydroxyl protons. Such groups can be utilized as endogenous CEST agents, which can further allow a non-invasive assessment of GAG concentration in vivo. For example, such assessment of GAG concentration can be performed using MRI techniques through a CEST contrast mechanism (“gagCEST”).

Osteoarthritis (“OA”) may be characterized by a loss of proteoglycan (“PG”) and a corresponding loss of GAG or other compounds. Exemplary embodiments of the present invention can be used, e.g., to demonstrate a sensitivity of gagCEST to GAG concentration variation in cartilage tissue. Such determination of GAG concentration in cartilage can be performed either ex vivo or in vivo, and could assist in a diagnosis of early stages of OA.

For example, —OH hydroxyl protons at δ=+1.0 ppm, among other labile protons, can be used to monitor GAG concentration in cartilage in vivo. Such —OH hydroxyl protons may be particularly useful, in part, because of their high concentration and fast exchange rate. Exemplary methods and systems which analyze such —OH hydroxyl protons using gagCEST techniques can provide useful clinical applications and basic research tools.

CEST techniques as applied to the —NH amide proton site of GAG may likely provide further information about GAG assessment. Factors such as, e.g., high saturation efficiency, specificity, low SAR, and non-invasiveness can render gagCEST particularly useful for assessing cartilage and IVDs.

The CEST technique can be used for the detection and imaging of residues carrying non-exchangeable protons as well. Here the mechanism is via cross-relaxation or the Nuclear Overhauser Effect (NOE) from these residues to water. This novel application can be named NOE saturation transfer or NOEST. Specifically for the application of GAG imaging it would be gagNOEST.

The foregoing merely illustrates the principles of the invention. Various modifications and alterations to the described embodiments will be apparent to those skilled in the art in view of the teachings herein. It will thus be appreciated that those skilled in the art will be able to devise numerous systems, arrangements, and methods which, although not explicitly shown or described herein, embody the principles of the invention and are thus within the spirit and scope of the invention. In addition, all publications and references referred to above are incorporated herein by reference in their entireties. It should be understood that the exemplary procedures described herein can be stored on any computer accessible medium, including a hard drive, RAM, ROM, removable discs, CD-ROM, memory sticks, etc., and executed by a processing arrangement which can be a microprocessor, mini, macro, mainframe, etc.

What is claimed is:

1. A computer-accessible medium for evaluating an anatomical region of interest including instructions thereon, wherein, when a processing arrangement executes the instructions, the processing arrangement is configured to perform procedures comprising:
determining one or more particular frequencies of cross-relaxation between at least one molecule and at least one particular compound;
determining a chemical exchange based on magnetic resonance data using a further frequency which is different from the one or more particular frequencies; and
deriving particular information about the anatomical region of interest based on the chemical exchange.

2. The computer-accessible medium according to claim 1, wherein the magnetic resonance data pertains to particular nuclei.

3. The computer-accessible medium according to claim 2, wherein the particular nuclei are exchangeable nuclei.

4. The computer-accessible medium according to claim 3, wherein the exchangeable nuclei are protons.

5. The computer-accessible medium according to claim 4, wherein the exchangeable protons are at least one of hydroxyl protons or amide protons.

6. The computer-accessible medium according to claim 1, wherein the chemical exchange is determined by measuring at least one of a chemical transfer or a magnetization transfer of the at least one molecule.

7. The computer-accessible medium according to claim 1, wherein the chemical exchange is determined using a chemical exchange saturation transfer pulse sequence applied to target the at least one molecule.

8. The computer-accessible medium according to claim 1, wherein the particular information is a concentration or a density of the at least one molecule.

9. The computer-accessible medium according to claim 1, wherein the anatomical region of interest is at least one of a knee, an intervertebral disc, a cornea, a cervix or a heart valve.

10. The computer-accessible medium according to claim 1, wherein the derivation procedure relates to at least one of osteoarthritis or intervertebral disc degeneration.

11. The computer-accessible medium according to claim 1, wherein the chemical exchange is determined using an offset relative to a water signal of the at least one compound.

12. The computer-accessible medium according to claim 11, wherein the offset is at least one region around −2.6 ppm, −1 ppm, +1 ppm, +2.6 ppm or +3.2 ppm.

13. The computer-accessible medium according to claim 2, wherein the nuclei are isotopes of at least one of phosphorus, xenon, carbon, sodium or fluorine.

14. The computer-accessible medium according to claim 8, wherein the concentration or the density is associated with at least one of glycosaminoglycan or proteoglycan.

15. The computer-accessible medium according to claim 1, further comprising generating an image of the anatomical region of interest.

16. The computer-accessible medium according to claim 15, wherein the image is a difference image.

17. The computer-accessible medium according to claim 16, wherein in the difference image there is a difference between two images relating to equal and opposite offsets.

18. The computer-accessible medium according to claim 1, wherein the at least one molecule is a macromolecule.

19. The computer-accessible medium according to claim 1, further comprising:
determining a frequency region in which chemical exchange and cross-relaxation do not interfere

20. A computer accessible medium for evaluating an anatomical region of interest including instructions thereon, wherein, when a processing arrangement executes the instructions, the processing arrangement is configured to perform procedures comprising:
selecting one or more particular frequencies of a cross-relaxation between at least one macromolecule and at least one particular compound;
determining at least one of a chemical transfer or a magnetization transfer from magnetic resonance data using at least one of the particular frequencies; and
deriving particular information about the anatomical region of interest from at least one of the chemical transfer or the magnetization transfer.

21. The computer-accessible medium according to claim 20, wherein the magnetic resonance data pertains to particular nuclei.

22. The computer-accessible medium according to claim 21, wherein the particular nuclei are exchangeable nuclei.

23. The computer-accessible medium according to claim 22, wherein the exchangeable nuclei are protons.

24. The computer-accessible medium according to claim 23, wherein the exchangeable protons are at least one of hydroxyl protons or amide protons.

25. The computer-accessible medium according to claim 20, wherein the chemical exchange is determined by measuring at least one of a chemical transfer or a magnetization transfer of the at least one molecule.

26. The computer-accessible medium according to claim 20, wherein the chemical transfer is determined using a chemical exchange saturation transfer pulse sequence applied to target the at least one molecule.

27. The computer-accessible medium according to claim 20, wherein the particular information is a concentration or a density of the at least one molecule.

28. The computer-accessible medium according to claim 20, wherein the anatomical region of interest is at least one of a knee, an intervertebral disc, a cornea, a cervix or a heart valve.

29. The computer-accessible medium according to claim 20, wherein the derivation procedure relates to at least one of osteoarthritis or intervertebral disc degeneration.

30. The computer-accessible medium according to claim 20, wherein the chemical transfer is determined using an offset relative to a water signal of the at least one compound.

31. The computer-accessible medium according to claim 30, wherein the offset is at least one region around −2.6 ppm, −1 ppm, +1 ppm, +2.6 ppm or +3.2 ppm.

32. The computer-accessible medium according to claim 21, wherein the nuclei are isotopes of at least one of phosphorus, xenon, carbon, sodium or fluorine.

33. The computer-accessible medium according to claim 27, wherein the concentration or the density is associated with at least one of glycosaminoglycan or proteoglycan.

34. The computer-accessible medium according to claim 20, further comprising generating an image of the anatomical region of interest.

35. The computer-accessible medium according to claim 34, wherein the image is a difference image.

36. The computer-accessible medium according to claim 35, wherein in the difference image is a difference between two images relating to equal and opposite offsets.

37. The computer-accessible medium according to claim 36, wherein the at least one molecule is a macromolecule.

38. The computer-accessible medium according to claim 20, further comprising:
determining a frequency region in which chemical exchange and cross-relaxation do not interfere
